(19) United States
(12) Patent Application Publication
(10) Pub. No.: US 2026/0314949 A1
(43) Pub. Date: Oct. 15, 2026

(54) BIOLOGICAL INFORMATION MEASUREMENT APPARATUS AND BIOLOGICAL INFORMATION MEASUREMENT METHOD

(71) Applicant: Asahi Kasei Microdevices Corporation, Tokyo (JP)

(72) Inventors: Takahide ONODA, Tokyo (JP); Shigeru IWASA, Tokyo (JP)

(21) Appl. No.: 19/687,092

(22) Filed: May 26, 2026

Related U.S. Application Data
(63) Continuation of application No. PCT/JP2024/040878, filed on Nov. 18, 2024.

(30) Foreign Application Priority Data
Nov. 29, 2023 (JP) 2023-202107

(51) Int. Cl. A61B 5/053; A61B 5/00

(52) U.S. Cl. CPC A61B 5/053; A61B 5/7225; A61B 5/725; A61B 5/7278

(57) ABSTRACT

Provided is a biological information measurement apparatus including: an AC signal supply unit that supplies an AC signal indicating a reference frequency between a pair of electrodes that contact a living body; a first high-pass filter that blocks, from a first signal corresponding to the AC signal, a frequency band lower than a first frequency band including the reference frequency; and a first amplification unit that amplifies a third signal, which is obtained by blocking, from a second signal output from the first high-pass filter, a second frequency band narrower than the first frequency band and including the reference frequency, and outputs the amplified third signal as a biological information signal indicating a time-varying component of impedance of the living body.

The contents of the following patent application(s) are incorporated herein by reference:
NO. 2023-202107 filed in JP on Nov. 29, 2023
NO. PCT/JP2024/040878 filed in WO on Nov. 18, 2024.

### BACKGROUND

### 1. Technical Field

The present invention relates to a biological information measurement apparatus and a biological information measurement method.

### 2. Related Art

Patent Document 1 describes a bioimpedance measurement apparatus that can be mounted on an implantable medical device. Patent Document 2 describes a biosignal measurement apparatus having a bioimpedance measurement unit. Patent Document 3 describes an electronic medical device for simultaneously measuring impedance and a biopotential signal.

### RELATED ART DOCUMENTS

### Patent Documents

Patent Document 1: Japanese Patent Application Publication No. 2012-157463

Patent Document 2: European Patent No. 3692908

Patent Document 3: European Patent No. 2294979

### BRIEF DESCRIPTION OF THE DRAWINGS

FIG. 1 is an example of a schematic diagram for explaining bioimpedance BioZ generated in a living body 300.

FIG. 2 is a block diagram illustrating an example of a configuration of a biological information measurement apparatus 10a.

FIG. 3A is an example of a conceptual diagram for explaining processing performed by a high-pass filter 12.

FIG. 3B is an example of a conceptual diagram for explaining processing performed by a mixer 22.

FIG. 3C is an example of a conceptual diagram for explaining processing performed by a low-pass filter 24.

FIG. 3D is an example of a conceptual diagram for explaining processing performed by a high-pass filter 26.

FIG. 3E is an example of a conceptual diagram for explaining processing performed by an amplification circuit 40.

FIG. 4 is an example of a flowchart of a biological information measurement method using the biological information measurement apparatus 10a.

FIG. 5 is an example of a block diagram illustrating a configuration of a biological information measurement apparatus 10b.

FIG. 6A is a circuit diagram illustrating an example of a specific configuration of a separator 28, the amplification circuit 40, and a multiplexer 42.

FIG. 6B is a circuit diagram illustrating another example of the specific configuration of the separator 28, the amplification circuit 40, and the multiplexer 42.

FIG. 7 is an example of a flowchart of a biological information measurement method using the biological information measurement apparatus 10b.

FIG. 8 is an example of a block diagram illustrating a configuration of a biological information measurement apparatus 10c.

FIG. 9A is an example of a conceptual diagram for explaining an operation of a high-pass filter 30.

FIG. 9B is an example of a conceptual diagram for explaining an operation of a notch filter 32.

FIG. 9C is an example of a conceptual diagram for explaining a signal VA5 output by the amplification circuit 40 by amplifying a signal VN.

FIG. 10 is an example of the flowchart of a biological information measurement method using the biological information measurement apparatus 10c.

FIG. 11A is a diagram illustrating an example of a configuration of an AC signal supply unit 50b.

FIG. 11B is a diagram illustrating an example of a configuration of an AC signal supply unit 50c.

FIG. 12 is a diagram illustrating an example of a configuration of an AC signal supply unit 50d.

FIG. 13 is a diagram illustrating an example of a modification in which, in the embodiment of FIG. 2, components other than a bioimpedance measurement unit 100a are replaced.

FIG. 14 is a block diagram illustrating an example of a configuration of a biological information measurement apparatus 10d.

### DESCRIPTION OF EXEMPLARY EMBODIMENTS

The present invention will be described below through embodiments of the invention, but the following embodiments do not limit the invention according to the claims. In addition, not all of the combinations of features described in the embodiments are essential to the solving means of the invention.

In the present specification, when referring to modified forms of a same element, for example, the element may be referred to with an alphabetic character appended thereto, such as a bioimpedance measurement unit 100a, a bioimpedance measurement unit 100b, and the like. On the other hand, these constituent elements may also be collectively referred to, for example, as a bioimpedance measurement unit 100 or the like.

A wristband-type controller, an electromyographic prosthetic hand, or the like is known that predicts a human motion by detecting a myoelectric potential generated immediately before a human performs an action and controls an object in accordance with the predicted human motion. An apparatus such as the wristband-type controller that detects a motion of a living body such as a human includes a biological sensor such as a myoelectric sensor, senses biological information such as a myoelectric potential or bioimpedance, and predicts biological information regarding the living body such as a motion of the living body.

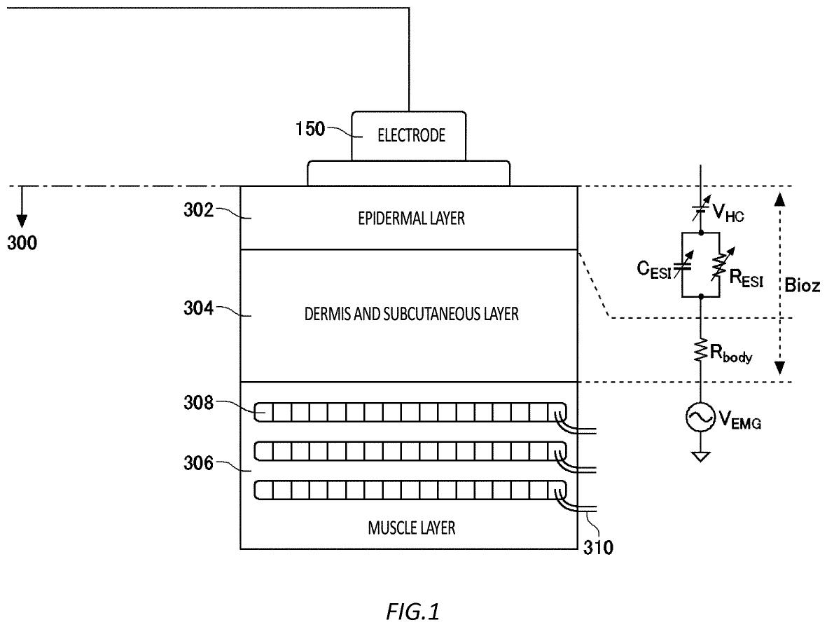

FIG. 1 is the example of a schematic diagram for explaining bioimpedance BioZ generated in a living body 300. The living body 300 includes an epidermal layer 302, a dermis and subcutaneous layer 304, and a muscle layer 306.

The living body 300 of the present embodiment is a human. However, the living body 300 may be another animal. When the living body 300 is another animal, some skin structures of the living body 300 may differ depending on the type of the living body 300.

In measurement of the bioimpedance BioZ, a pair of electrodes 150 are brought into contact with and fixed to the living body 300, and a constant current is applied between the electrodes. In this case, by reading a potential difference generated between the electrodes 150, the bioimpedance BioZ based on body composition of the living body 300 can be read.

Further, for example, when the living body 300 activates muscles, an action potential is generated in the muscle layer 306 of the living body 300 due to electrical excitation of cells. In measurement of such an action potential, impedance attributable to the epidermal layer 302 and the dermis and subcutaneous layer 304 affects the measurement.

The epidermal layer 302 is the epidermis of the living body 300. For example, when the living body 300 is a human, the epidermal layer 302 is a portion having an average thickness of about 0.2 mm in skin at portions other than a palm of a hand or a sole of a foot.

In measurement of the bioimpedance BioZ by the electrode 150, the epidermal layer 302 contributes as a half-cell potential VHC and a variable resistance RESI and a variable capacitance CESI connected in series with the half-cell potential VHC. The half-cell potential VHC is an electrostatic potential generated at a portion where the electrode 150 and the epidermal layer 302 are in contact. A component of the half-cell potential VHC contributes, in measurement of the bioimpedance BioZ, for example, as a component at a frequency of 20 Hz or lower. In the bioimpedance BioZ, a contribution of contact impedance between the electrode 150 and the skin of the living body 300, that is, contact impedance between the electrode 150 and the epidermal layer 302, is large. Further, the epidermal layer 302 contributes as the variable resistance RESI and the variable capacitance CESI connected in series with the half-cell potential VHC. The variable resistance RESI and the variable capacitance CESI vary significantly depending on the condition of the skin surface. This appears, in the bioimpedance BioZ, as a variation in the contact impedance between the electrode 150 and the skin of the living body 300. When the skin surface is in a dry state, these impedances increase by about ten times, and thus are expressed as a variable resistance and a variable capacitance in an equivalent circuit.

The dermis and subcutaneous layer 304 is a layer such as a dermal layer, subcutaneous tissue, and fascia. In measurement of the bioimpedance, the dermis and subcutaneous layer 304 contributes as a resistance Rbody. In the dermis and subcutaneous layer 304, the dermal layer is a site in the skin through which capillaries, lymphatic vessels, nerves, and the like pass, and is a portion formed inside the epidermal layer 302. For example, when the living body 300 is a human, the dermal layer is a portion having a thickness of about 2 mm on average. In the dermis and subcutaneous layer 304, the subcutaneous tissue is formed further inside the dermal layer. The subcutaneous tissue is a portion that supports the epidermal layer 302 and the dermal layer, and is a portion having a thickness of about 2 mm to about 9 mm on average. The subcutaneous layer mainly contains fat cells and includes large blood vessels and the like. Furthermore, the dermis and subcutaneous layer 304 includes fascia between the subcutaneous tissue and the muscle layer 306. The fascia has a thickness of about 1 mm and is a portion that generates an electrical resistance, although not as large as the skin. The resistance Rbody of the dermis and subcutaneous layer 304 is a resistance value obtained by adding the electrical resistances of the plurality of these layers.

In the end, in impedance measurement of the living body 300, what contributes as bioimpedance BioZ is the variable resistance RESI and the variable capacitance CESI connected in parallel, and the resistance Rbody.

The muscle layer 306 is a layer including a plurality of muscle fibers 308. The muscle fiber 308 is tissue that is activated by an electrical signal transmitted along a nerve 310. When the muscle fiber 308 moves, an action potential is generated in the muscle fiber 308. In the muscle layer 306, a potential VEMG (the composite action potential VEMG) obtained by adding (combining) action potentials of a plurality of muscle fibers is generated with activity of a plurality of muscles. In measurement of a myoelectric signal, measurement of such a composite action potential VEMG is performed.

The myoelectric signal is an example of a “biopotential signal”. The myoelectric signal may have a peak of amplitude in a frequency band higher than 20 Hz and up to 4 kHz, for example. As another example, the biopotential signal may be a signal indicating a potential generated by the activity of the living body such as cardiac activity, brain waves, or eye movements. Note that these biopotential signals are examples, and the biopotential signal is not limited to these signals as long as the biopotential signal is a signal based on the action potential of the living body 300 generated by activity of the living body 300.

For example, when the biopotential signal is an electrocardiographic signal, the electrocardiographic signal may have an amplitude peak in a frequency band higher than 0.05 Hz and up to 100 Hz. As another example, when the biopotential signal is an electroencephalographic signal, the electroencephalographic signal may have an amplitude peak in a frequency band higher than 0.5 Hz and up to 70 Hz. As still another example, when the biopotential signal is an eye movement signal, the eye movement oscillation may have an amplitude peak in a frequency band higher than 0.05 Hz and up to 20 Hz. In the following, description will be given by taking, as an example, a case where the biopotential signal is a myoelectric signal in particular. However, the biopotential signal is not limited to the myoelectric signal, and may be another biopotential signal.

Here, in measurement of the bioimpedance BioZ, when the living body 300 moves, a motion artifact (MA) generated at a contact point between the electrode 150 and the living body 300 may occur as measurement noise due to the electrode 150 being displaced with respect to the living body 300 or the living body 300 vibrating.

The MA generated when the living body 300 moves often has a peak at lower than 20 Hz, for example, when the living body 300 is a human. The influence of such an MA can be blocked by a high-pass filter described later. However, even when a frequency band in which a peak of MA appears is a frequency band of 20 Hz or lower, if the peak value of MA is large, the influence of MA trailing from the peak may appear in a frequency band of 20 Hz or higher.

On the other hand, vibration generated in the living body 300 is, for example, vibration generated in a moving body such as a train, a bus, or an airplane in which a human is riding, or the like. MA generated while the living body 300 is riding in the moving body can be directly mixed as noise into, for example, a frequency band of 50 Hz or higher and lower than 200 Hz. In this case, the MA is known to contribute as a variation in a capacitive component, and contributes as a component proportional to a time-varying component of CESI. Furthermore, a variation in bioimpedance BioZ due to MA may have a frequency higher than the predetermined frequency (for example, 20 Hz).

Therefore, in the measurement of the variable capacitance CESI, by blocking a component in an appropriate frequency range and amplifying the blocked component, it is possible to read a variation in capacitive element proportional to the MA, and eventually, it is possible to read a variation in bioimpedance due to the MA. In the following, a configuration of a biological information measurement apparatus capable of reading such a variation of variable capacitance CESI will be described in detail.

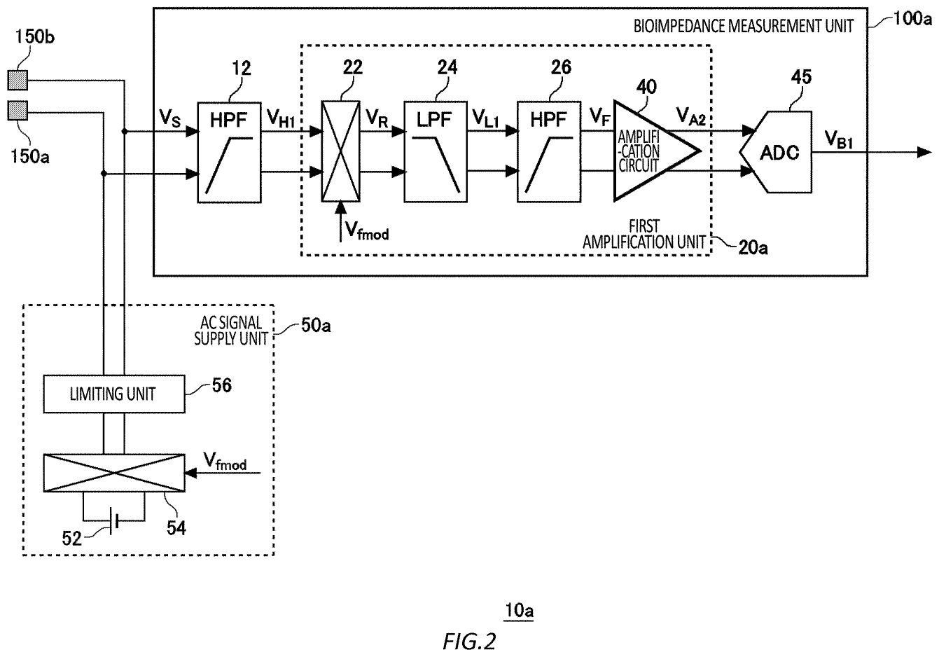

FIG. 2 is a block diagram illustrating an example of a configuration of a biological information measurement apparatus 10a. The biological information measurement apparatus 10a includes an AC signal supply unit 50, a bioimpedance measurement unit 100a, and electrodes 150a and 150b.

The AC signal supply unit 50 supplies an AC signal of a rectangular wave that is alternately switched on and off between a pair of electrodes 150a and 150b that contact a living body. That is, the AC signal supply unit 50 supplies the AC signal modulated at a reference frequency between the pair of electrodes 150a and 150b that contact the living body. The AC signal supply unit 50 includes a power supply 52, a chopper 54, and a limiting unit 56.

The power supply 52 is, as an example, a DC power supply such as a battery.

The chopper 54 replaces a DC voltage supplied from the power supply 52 with a signal that is turned on and off at a constant cycle. That is, the chopper 54 outputs a rectangular wave in which an output potential is switched at a constant cycle. The chopper 54 may receive a reference frequency signal Vfmod in order to switch the potential of the rectangular wave at a cycle of a reference frequency fmod (for example, a frequency of 10 kHz or higher and 100 kHz or lower). In this case, the chopper 54 functions as a “voltage output circuit” that switches a voltage from the DC power supply at the reference frequency fmod and outputs a voltage signal. The chopper 54 includes, for example, a switching element that is turned on and off at a cycle 1/fmod.

The limiting unit 56 outputs, as the AC signal flowing from the AC signal supply unit 50 through the electrodes 150a and 150b, a current signal according to the reference frequency fmod by application of the voltage signal of the rectangular wave output from the chopper 54, and in this case, the limiting unit 56 limits a magnitude of a current flowing through the living body 300. The limiting unit 56 includes, for example, a resistor.

The bioimpedance measurement unit 100a measures the bioimpedance BioZ by reading a signal Vs indicating a potential difference generated between the pair of electrodes 150a and 150b that contact the living body 300 when a rectangular-wave current is caused to flow through the pair of electrodes 150a and 150b. The bioimpedance measurement unit 100a includes a high-pass filter 12, a first amplification unit 20a, and an analog to digital converter (ADC) 45.

The high-pass filter 12 blocks, from the signal Vs, a frequency band (for example, a frequency band of 100 Hz or lower; that is, in this example, a cutoff frequency fC1 of the high-pass filter 12=100 Hz) lower than a first frequency band including the reference frequency fmod (for example, a frequency band of 28 kHz or higher and 36 kHz or lower), and outputs a signal VH1. The reference frequency fmod corresponds to a median value of the first frequency band. Therefore, the example shown in the present embodiment is an example in which the reference frequency fmod=32 kHz, and the first frequency band is an example set to a range of the reference frequency fmod±4 kHz that is the median value.

Here, a frequency of a signal generated by the half-cell potential VHC when measuring the bioimpedance BioZ is lower than a certain frequency (for example, 20 Hz). Therefore, filtering by the high-pass filter 12 corresponds to blocking the contribution of the half-cell potential VHC in the signal VS. That is, the cutoff frequency fc1 of the high-pass filter 12 is lower than the reference frequency fmod and higher than a frequency of a potential component generated by the half-cell potential VHC. The frequency of the potential component generated by the half-cell potential VHC may be determined based on, for example, an average value of measured values measured in advance by experiments or the like.

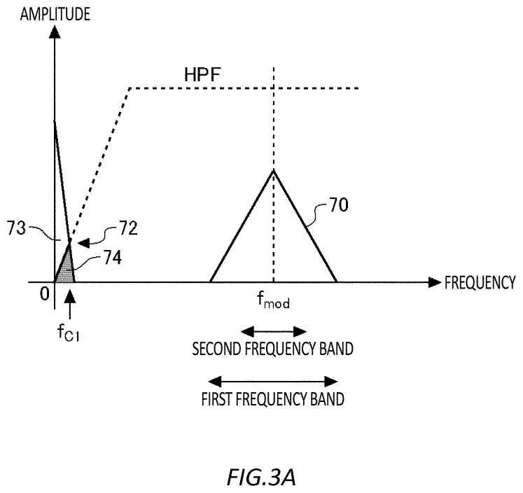

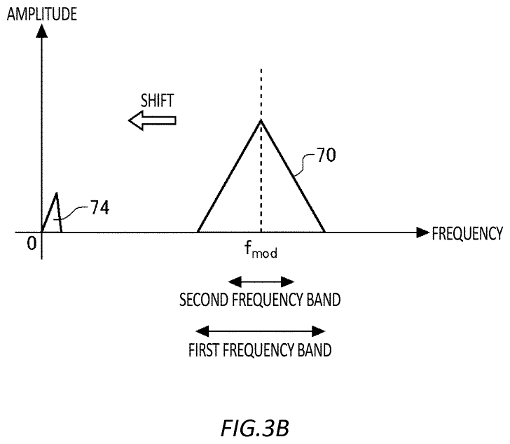

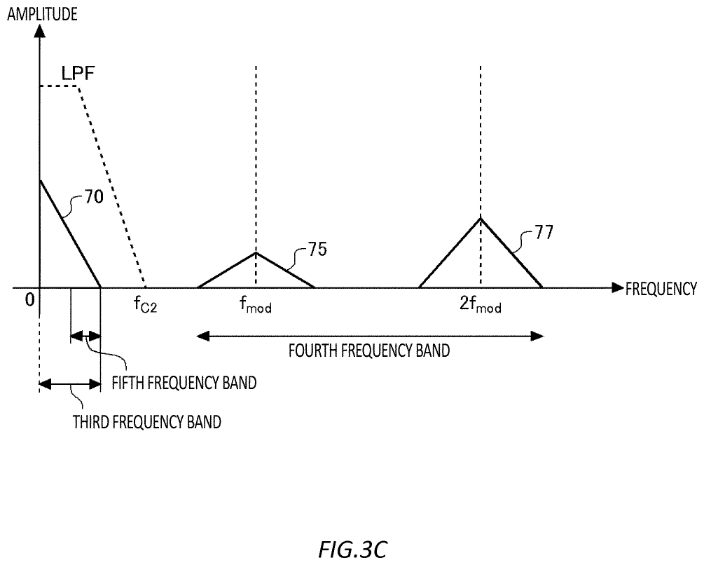

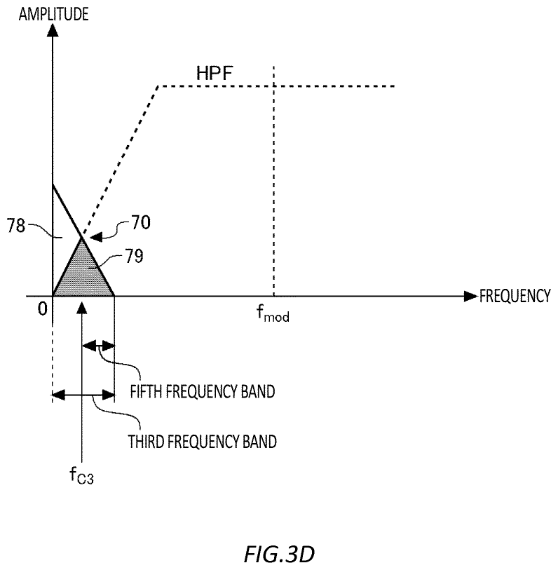

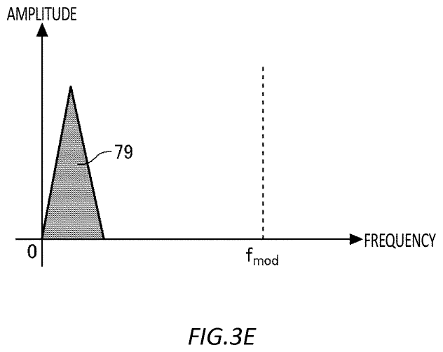

The processing performed by the high-pass filter 12 is specifically illustrated in FIG. 3A. FIG. 3A is an example of a conceptual diagram for explaining the processing performed by the high-pass filter 12. In FIGS. 3A to 3E, an outline of signals output by processing performed by respective constituent elements of the first amplification unit 20a is described with a horizontal axis representing a frequency and a vertical axis representing a magnitude of a signal amplitude. In particular, in FIG. 3A, an outline of the signal VH1 output after the processing performed by the high-pass filter 12 is described.

The signal VS supplied to the high-pass filter 12 is a signal indicating a potential difference generated between the pair of electrodes 150a and 150b that contact the living body 300. The signal VS includes an action potential component 72 appearing in a vicinity of the zero frequency and a bioimpedance component 70 appearing in a vicinity of the reference frequency fmod.

The bioimpedance component 70 has, in the vicinity of the reference frequency fmod, the component of the average value of the bioimpedance BioZ, and includes, as a component other than the component of the average value, a time-varying component of the bioimpedance BioZ. In the drawing, a frequency band in which the bioimpedance component 70 appears is shown as the first frequency band, and the frequency band in which an average component of the bioimpedance component 70 is included is shown as a second frequency band (for example, a frequency band of 31.98 kHz or higher and 32.02 kHz or lower). That is, the time-varying component of the bioimpedance BioZ is shown as a portion obtained by excluding, from the bioimpedance component 70 included in the first frequency band, a component included in the second frequency band.

On the other hand, the action potential component 72 includes a half-cell potential component 73 from the half-cell potential VHC in a band in a frequency range in the vicinity of the zero frequency. Furthermore, the action potential component 72 includes, in a band other than a range including the half-cell potential component 73, a component in which action potentials from respective ones of a plurality of biological tissues (for example, muscle fibers 308) constituting the living body 300 are combined.

Here, in measurement of the bioimpedance BioZ, it is known that the time-varying component of the bioimpedance BioZ includes a component proportional to the motion artifact. In addition, the time-varying component of the bioimpedance component 70 becomes a signal with a very small amplitude, for example, having an amplitude of about one-several hundredth compared to an amplitude due to the contribution of the action potential component 72 in the vicinity of the zero frequency and an average component of the bioimpedance component 70 appearing in the vicinity of the reference frequency fmod.

The bioimpedance component 70 measured as the analog signal can be converted into a digital signal by an ADC. Each ADC has a dynamic range that is a numerical value determined by a ratio between a maximum value and a minimum value of a signal that can be processed. When amplification is performed, without performing appropriate blocking of a signal by frequency, for the purpose of amplifying the time-varying component of the bioimpedance component 70, the contribution of the action potential component 72 and the contribution of the average component of the bioimpedance component 70 appearing in the vicinity of the reference frequency fmod increase, and requirements for the dynamic range of the ADC 45 may become stringent.

For example, in the bioimpedance measurement apparatus of Patent Document 1, an average component of the impedance is subtracted by adjusting a common voltage of the impedance signal amplification unit while monitoring the output range of the ADC. Also, for example, in the biological signal measurement apparatus having the bioimpedance measurement unit of Patent Document 2, the average component of the impedance is subtracted by supplying, to the impedance signal amplification unit, a correction current based on an average value of ADC output. In feedback control performed as in Patent Document 1 and Patent Document 2, it may be impossible to follow variations in the average value of the bioimpedance caused by electrode displacement, and design requirements for the dynamic range of the ADC may also become stringent.

Patent Document 3 describes the electronic medical device for simultaneously measuring impedance and a biopotential signal. However, such an electronic medical device does not have a configuration for extracting the time-varying component of the bioimpedance component 70 during the impedance measurement.

Therefore, it is desired to simultaneously measure the bioimpedance BioZ and the biopotential signal, and to extract only the time-varying component of the bioimpedance component 70 with higher accuracy. If only the time-varying component of the bioimpedance component 70 having a small amplitude can be extracted with high accuracy, the requirements for the dynamic range of the ADC for performing AD conversion of the time-varying component is relaxed. The dynamic range of the ADC is, for example, a resolution indicating a ratio of a maximum value of an amplitude of a sine wave handled in AD conversion with respect to an error including a quantization error (signal-to-noise ratio).

Therefore, in such an apparatus, even when the contact impedance between the electrodes 150a and 150b and the living body 300 changes steeply due to vibration occurring in the living body 300, it is desired to extract a noise component with high accuracy and thereby exclude an influence of MA mixed therein from the bioimpedance to be measured. The bioimpedance measurement unit 100a of the present embodiment enables, by including the high-pass filter 12 and the first amplification unit 20a, more accurately excluding, from the signal VS, components other than the time-varying component of the bioimpedance component 70.

The high-pass filter 12 blocks a frequency band lower than the cutoff frequency fC1 in order to block an influence due to the half-cell potential component 73. A potential that can remain in a vicinity of the action potential component 72 after blocking by the high-pass filter 12 is a residual component 74. The residual component 74 becomes a component including the contribution of the biopotential signal VEMG when the cutoff frequency fC1 is set sufficiently low and the contribution of the half-cell potential component 73 is blocked from the action potential component 72. However, in the bioimpedance measurement unit 100a, since measurement itself of the biopotential signal VEMG is not performed, in the high-pass filter 12, the cutoff frequency fC1, a transition width, and the like may be set so that the residual component 74 does not remain. The signal VH1 output from the high-pass filter 12 includes the bioimpedance component 70 and the residual component 74.

Note that the high-pass filter 12 may be a so-called active filter configured by connecting a resistor and a capacitor to a terminal to which a voltage to be amplified is input in an inverting amplifier circuit or a non-inverting amplifier circuit configured with an Op-Amp. When such an active filter is used, blocking of a signal having a frequency lower than the cutoff frequency fC1 and amplification of the signal are performed together. Thus, by using the active filter as the high-pass filter 12, when it is desired to obtain a signal having a large amplitude as the signal VH1 after blocking, the amplified signal can be output as the signal VH1. Further, when the active filter is used, the number of stages of resistors and capacitors included in the active filter is not limited. That is, the order of the filter is not limited, and the filter having a desired order may be used.

The first amplification unit 20a in FIG. 2 blocks, from the signal VH1 output from the high-pass filter 12, the second frequency band that includes the reference frequency fmod and is narrower than the first frequency band. In the first frequency band, the contribution from the average value of bioimpedance BioZ appears in the vicinity of the reference frequency fmod, and the contribution of the time-varying component of bioimpedance BioZ appears at a position away from the reference frequency fmod. Therefore, blocking of the second frequency band by the first amplification unit 20a corresponds to blocking the contribution of the average component of the bioimpedance BioZ and extracting the contribution of the time-varying component of the bioimpedance BioZ. The first amplification unit 20a includes a mixer 22, a low-pass filter 24, a high-pass filter 26, and an amplification circuit 40.

FIG. 3B is an example of a conceptual diagram for explaining processing performed by the mixer 22. The signal VH1 is input from the high-pass filter 12 to the mixer 22 of the first amplification unit 20a. The mixer 22 converts the signal VH1 into a signal VR in a third frequency band lower than the first frequency band (for example, a frequency band of 4 kHz or lower) by mixing, into the signal VH1, a signal indicating the reference frequency fmod.

Specifically, the mixer 22 mixes the reference frequency signal Vfmod into the signal VH1 and outputs the signal VR obtained by shifting the signal VH1 to the vicinity of the zero frequency. Processing called “shift” illustrated in FIG. 3B indicates processing by this mixer 22. The mixer 22 acts on the signal VH1 including the bioimpedance component 70 and the residual component 74 to shift these signals to the vicinity of the zero frequency. The third frequency band is a band having, with the zero frequency as a lower limit value, a bandwidth including the bioimpedance component 70. The bandwidth including the bioimpedance component 70 may be set based on, for example, the average component of the bioimpedance component measured in advance by experiments or the like.

After this mixing processing, most of the bioimpedance component 70 and the residual component 74 are shifted to the vicinity of the zero frequency, and a part of the signals remains and appears in the vicinity of the reference frequency fmod and in a vicinity of a frequency 2fmod that is twice the reference frequency fmod. FIG. 3C shows, in an emphasized size for explanation, signals appearing in the vicinity of the reference frequency fmod and in the vicinity of the frequency 2fmod that is twice the reference frequency fmod. By shifting the bioimpedance component 70 and the residual component 74 to the vicinity of the zero frequency, signal processing for extracting the time-varying component of the bioimpedance BioZ after the mixer 22 can be implemented in a low frequency band. Therefore, processing for extracting the time-varying component of the bioimpedance BioZ can be implemented with lower power consumption than processing in a high frequency band.

The low-pass filter 24 in FIG. 2 blocks, from the signal VR, a fourth frequency band that is higher than the third frequency band and includes a reference frequency band fmod (for example, a frequency band of 10 kHz or higher). Processing performed by the low-pass filter 24 on the signal VR is specifically illustrated in FIG. 3C.

FIG. 3C is an example of a conceptual diagram for explaining the processing performed by the low-pass filter 24. A lower-limit frequency of the fourth frequency band (cutoff frequency fC2; in the present embodiment, for example, the cutoff frequency fC2 may be set to 6 kHz) may be predetermined based on the reference frequency band fmod within a range not including the third frequency band.

The third frequency band is a frequency band in which the bioimpedance component 70 of the signal VH1 appears after being shifted to the vicinity of the zero frequency. The fourth frequency band is a frequency band including both the shift residual component 75 appearing in the vicinity of the reference frequency fmod by mixing by the mixer 22 and a high-frequency shift component 77 appearing in the vicinity of the frequency 2fmod that is twice the reference frequency fmod. The low-pass filter 24 has the cutoff frequency fC2 that is higher than the third frequency band and lower than the fourth frequency band.

A signal VF corresponds to a signal obtained by shifting a component of the signal VH1 that is within the first frequency band and outside the second frequency band. Therefore, the signal VF is a signal corresponding to the component of the signal VH1 that is within the first frequency band and outside the second frequency band.

The low-pass filter 24 blocks, for the signal VR, a frequency band higher than the cutoff frequency fC2, and outputs a signal VL1. Thus, the low-pass filter 24 functions as an anti-aliasing filter that blocks a high-frequency contribution that may be folded back from the ADC 45. Note that, in FIG. 3C, a fifth frequency band is shown as a frequency band included in the third frequency band, and this frequency band will be described below with reference to FIG. 3D.

Next, the high-pass filter 26 in FIG. 2 blocks, from the signal VL1 output from the low-pass filter 24, a frequency band lower than a cutoff frequency fC3 within the third frequency band, and outputs the signal as the signal VF. The signal VF output by the high-pass filter 26 is illustrated in FIG. 3D. FIG. 3D is an example of a conceptual diagram for explaining processing performed by the high-pass filter 26. In FIG. 3D, an average component of the bioimpedance component 70 shifted to the vicinity of the zero frequency appears in a band particularly close to the zero frequency within the third frequency band.

Therefore, the high-pass filter 26 blocks, from the bioimpedance component 70 shifted to the vicinity of the zero frequency, a signal in a frequency band lower than the cutoff frequency fC3 (for example, 20 Hz). The contribution of the average component of the bioimpedance component 70 appears in a frequency band 78, which is particularly close to the zero frequency in the third frequency band, even after the bioimpedance component 70 is shifted to the vicinity of the zero frequency. The high-pass filter 26 blocks the contribution of the average component of the bioimpedance component 70 by blocking the frequency band 78 lower than the cutoff frequency fC3. In this case, a contribution of a signal obtained by shifting the residual component 74 of the action potential component 72 to the zero frequency band is also included in the frequency band 78. The high-pass filter 26 also blocks the contribution from the residual component 74 by blocking the frequency band 78.

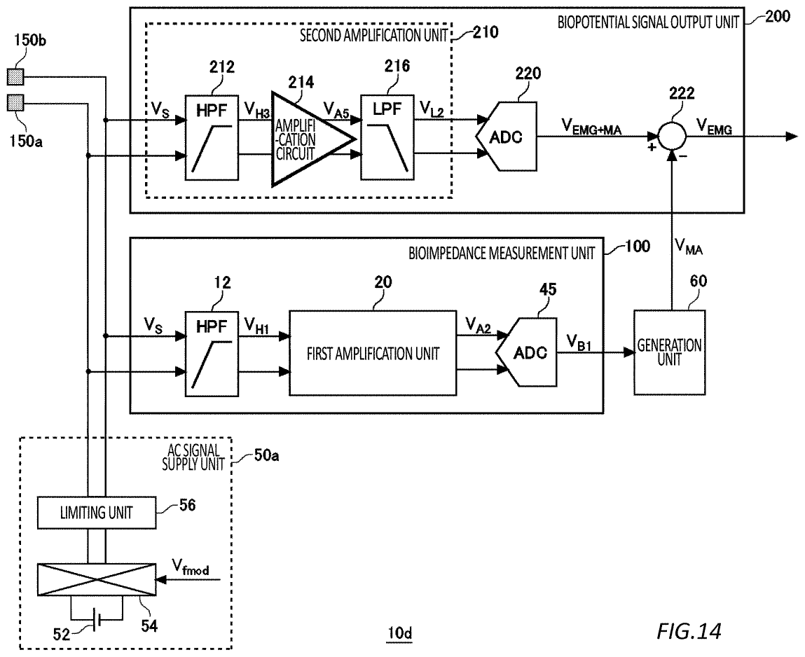

Therefore, the component of the signal VF remaining after the high-pass filter 26 performs blocking corresponds to a frequency band including a time-varying component 79 of the bioimpedance component. The cutoff frequency fC3 may be set to a frequency similar to a cutoff frequency of a high-pass filter 212 described later with reference to FIG. 14. In this case, the frequency band that is the cutoff frequency fC3 or higher and lower than an upper limit of the third frequency band (for example, 20 Hz or higher and lower than 4 kHz) is an example of the fifth frequency band described later. Note that the cutoff frequency fC3 of the high-pass filter 26 may be determined based on, for example, the average component of the bioimpedance component 70 measured in advance by experiments or the like.

The amplification circuit 40 of FIG. 2 amplifies the signal VF and outputs a signal VA2 indicating the time-varying component of the bioimpedance BioZ of the living body 300. FIG. 3E illustrates the signal VA2 output from the amplification circuit 40. FIG. 3E is an example of a conceptual diagram for explaining processing performed by the amplification circuit 40. The amplification circuit 40 amplifies the time-varying component 79 of the bioimpedance and outputs the signal VA2 corresponding to the component of the time-varying portion of the bioimpedance.

Note that as the low-pass filter 24 or the high-pass filter 26, an active filter configured by combining an Op-Amp, a resistor, and a capacitor and capable of executing both blocking processing and amplification processing may be used. In this case, the amplification circuit 40 may be omitted. In addition, for the active filter used in this case, a filter of a desired order may be used.

The ADC 45 of FIG. 2 AD (analog-to-digital) converts the signal VA2, which is an analog signal, and outputs a signal VB1, which is a digital signal of the time-varying component 79 of the bioimpedance component 70, and the signal VA2 or the signal VB1 obtained by AD-converting the signal VA2 is an example of a “biological information signal” indicating the time-varying component 79 of the bioimpedance component 70 of the living body 300.

In a process of acquiring the signal VA2 from the signal VS, contributions other than the time-varying component of the bioimpedance component 70 are excluded by processing of the high-pass filter 12, the mixer 22, the low-pass filter 24, the high-pass filter 26, and the like. Therefore, in the signal VA2, a contribution of a large-amplitude component other than the time-varying component of the bioimpedance component 70 is excluded, and the requirement for the dynamic range of the ADC 45 is relaxed. Thus, the biological information measurement apparatus 10a can output the signal VB1 obtained by digitally converting a component proportional to the motion artifact while an amplitude of a signal to be subjected to AD conversion relaxes the requirement for the dynamic range of the ADC 45. When the ADC 45 for which the requirement for the dynamic range is relaxed is used, the ADC with low power consumption and a small area (small footprint) can be used. Thus, an IC in which the ADC 45 is provided (for example, the IC may be configured to include the entirety of the biological information measurement apparatus 10a) can be realized with low power consumption and a small-area configuration.

In addition, due to an effect of the mixer 22, the frequency band of the signal VA2 that the ADC 45 AD-converts is shifted to a low frequency band in the vicinity of the zero frequency. Thus, power consumption for the ADC 45 to perform AD conversion can be reduced.

The high-pass filter 12 is an example of a “first high-pass filter”. The signal VS is an example of a “first signal”, the signal VH1 is an example of a “second signal”, and the signal VF is an example of a “third signal”.

Also, the mixer 22 is an example of a “first mixer”, and the low-pass filter 24 is an example of a “first low-pass filter”. The high-pass filter 26 is an example of a “second high-pass filter”.

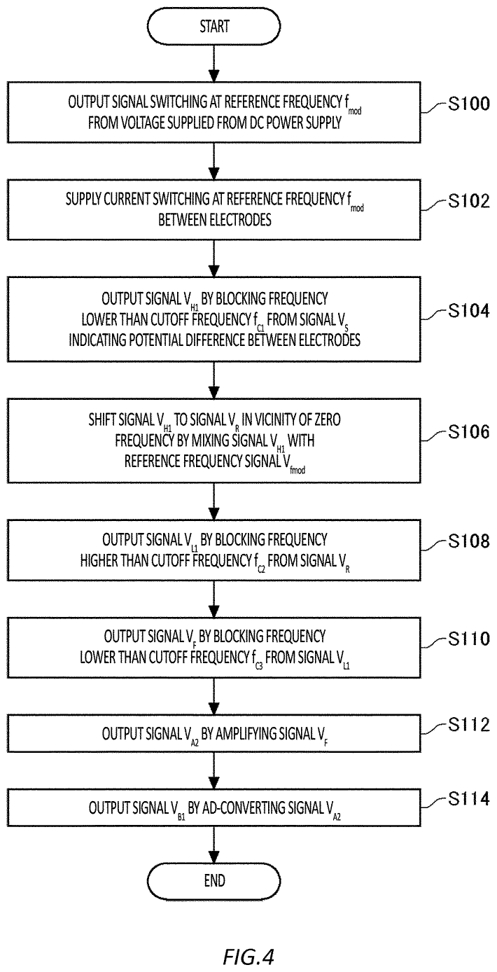

FIG. 4 is the example of a flowchart of a biological information measurement method using the biological information measurement apparatus 10a. The biological information measurement method using the biological information measurement apparatus 10a includes steps S100 to S114.

The chopper 54 outputs, from a DC voltage supplied from the power supply 52 that is a DC power supply, a signal whose voltage level (potential) is switched at the reference frequency fmod (S100). The limiting unit 56 supplies, in accordance with the voltage of the rectangular wave output from the chopper 54, a current between electrodes whose magnitude is switched at the reference frequency fmod (S102).

The bioimpedance measurement unit 100a reads the signal Vs indicating the potential difference generated between the pair of electrodes 150a and 150b that contact the living body 300 when a rectangular-wave current is applied to the pair of electrodes 150a and 150b. The high-pass filter 12 blocks, from the signal VS, a frequency lower than the cutoff frequency fmod and outputs the signal VH1 (S104). The mixer 22 mixes the signal VH1 with the reference frequency signal Vfmod to shift the signal VH1 to the signal VR in the vicinity of the zero frequency (S106).

The low-pass filter 24 blocks, from the signal VR, a frequency higher than the cutoff frequency fC2 and outputs the signal VL1 (S108). The high-pass filter 26 blocks, from the signal VL1, a frequency lower than the cutoff frequency fC3 and outputs the signal VF (S110).

The amplification circuit 40 amplifies the signal VF and outputs the signal VA2 (S112). The ADC 45 performs AD (analog-to-digital) conversion on the signal VA2 and outputs the signal VB1 (S114). As described above, the biological information measurement method using biological information measurement apparatus 10a enables the time-varying component of the bioimpedance BioZ including a component proportional to the motion artifact to be extracted with high accuracy by an apparatus including the IC with low power consumption and a small area.

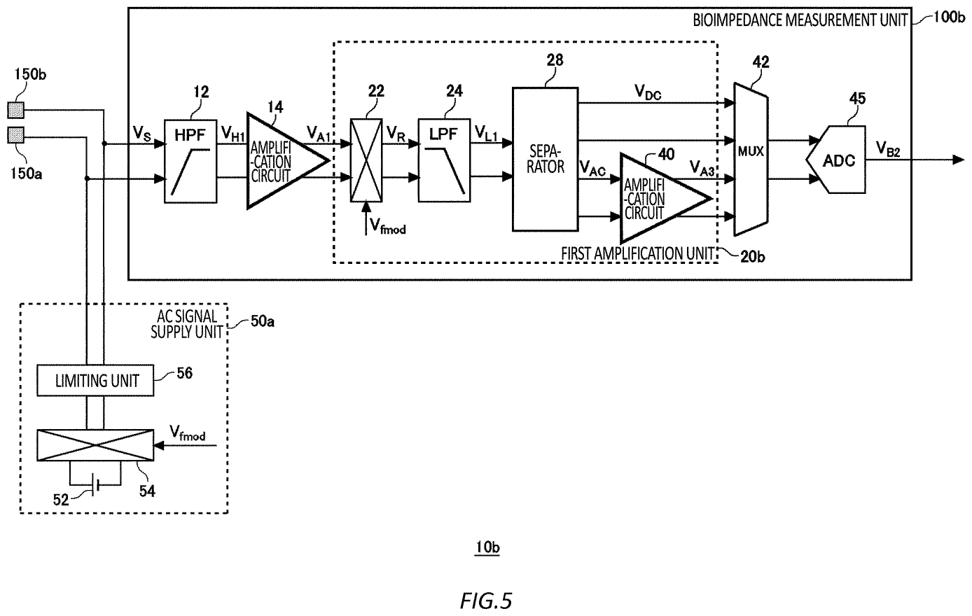

FIG. 5 is an example of a block diagram illustrating a configuration of the biological information measurement apparatus 10b. The biological information measurement apparatus 10b differs from biological information measurement apparatus 10a in that the biological information measurement apparatus 10b includes the bioimpedance measurement unit 100b.

In the following, description will be given focusing mainly on differences between the bioimpedance measurement unit 100a described with reference mainly to FIG. 2 and the bioimpedance measurement unit 100b of FIG. 5. The bioimpedance measurement unit 100b includes the high-pass filter 12, a first amplification unit 20b, the amplification circuit 40, the multiplexer 42, and the ADC 45.

Therefore, the bioimpedance measurement unit 100b differs from the bioimpedance measurement unit 100a in that the bioimpedance measurement unit 100b includes an amplification circuit 14, the first amplification unit 20b, and the multiplexer 42, and also differs in points such as a connection relationship among the first amplification unit 20b, the multiplexer 42, and the ADC 45.

The bioimpedance measurement unit 100b measures, similarly to the bioimpedance measurement unit 100a, the signal VS based on the potential difference generated between the electrodes 150a and 150b that contact the living body 300.

The amplification circuit 14 amplifies the signal VH1 and outputs a signal VA1. As a result, the contribution of the bioimpedance component 70 in the first frequency band in the signal VS and the contribution of the residual component 74 obtained by excluding the half-cell potential component 73 from the action potential component 72 are amplified. Here, the high-pass filter 12 can also be configured to be able to execute both amplification processing by the amplification circuit 14 and blocking processing by the high-pass filter, by being configured with an active filter using an Op-Amp, a resistor, and a capacitor. Thus, when the high-pass filter 12 is configured with an active filter, or when the amplitude of the signal VH1 has a magnitude sufficient to perform processing in a subsequent circuit, the amplification circuit 14 may be omitted, similarly to the biological information measurement apparatus 10a.

In the bioimpedance measurement unit 100b, since the configuration of the first amplification unit 20b differs from that of the first amplification unit 20a, a method by which the first amplification unit 20b removes the average component of the bioimpedance component 70 from the signal VA1 also differs from that of the bioimpedance measurement unit 100a. The first amplification unit 20b includes the mixer 22, the low-pass filter 24, a separator 28, and the amplification circuit 40.

In the first amplification unit 20b, a point that the mixer 22 outputs, from the signal VA1, the signal VR shifted to the vicinity of the zero frequency, and a point that the low-pass filter 24 blocks the signal VR and outputs the signal VL1 are the same as those of the first amplification unit 20a described with reference to FIG. 2. In the first amplification unit 20b, among VL1, a component close to the zero frequency in the third frequency band (a DC component; for example, a component of lower than 20 Hz) and another component (for example, a component in a frequency band higher than 20 Hz) are separated.

The separator 28 separates the signal VR into a signal VDC of a component in a frequency band lower than the first frequency (for example, 20 Hz) within the third frequency band and a signal VAC of a component in a frequency band higher than the first frequency. Specifically, the separator 28 separates, in the third frequency band of the signal VR, the signal VDC of the DC component that is a signal closer to the vicinity of the zero frequency and the signal VAC of the AC component that is another component in the third frequency band. As described with reference to FIG. 2, a signal having a component proportional to the motion artifact within the bioimpedance component 70 of the living body appears as the time-varying component of the bioimpedance component 70. The signal VAC corresponds to the AC component of the signal VR obtained by shifting the bioimpedance component 70 to the vicinity of the zero frequency band, and includes the time-varying component of the bioimpedance component 70.

The separator 28 outputs the signal VDC of the DC component of the signal VR to the ADC 45 via the multiplexer 42. On the other hand, the separator 28 outputs the signal VAC to the amplification circuit 40 in order to input the signal VAC to the ADC 45 after amplifying the signal VAC. The signal VAC is another example of the “third signal”.

The amplification circuit 40 amplifies the signal VAC and outputs a signal VA3 to the multiplexer 42. Since the amplitude of the signal VAC is smaller than that of the signal VDC, amplification by the amplification circuit 40 makes it possible to amplify the time-varying component of the bioimpedance component 70 to a signal having an amplitude that can be processed as a digital signal, and furthermore makes it possible to adjust a difference in amplitude between the signal VAC and the signal VDC.

The multiplexer 42 selectively outputs the signal VA3 or the signal VDC to the ADC 45. For example, the multiplexer 42 of the present embodiment outputs a differential signal based on the signal VA3 to the ADC 45. Here, the multiplexer 42 may output the signal VA3 and the signal VDC to the ADC 45, for example, in a time-division manner.

The ADC 45 performs AD (analog-to-digital) conversion on the signal output from the multiplexer 42 and outputs a signal VB2. The signal VA3, or the signal VB2 obtained by AD-converting the signal VA3, is another example of the “biological information signal” indicating the time-varying component of the bioimpedance BioZ of the living body 300.

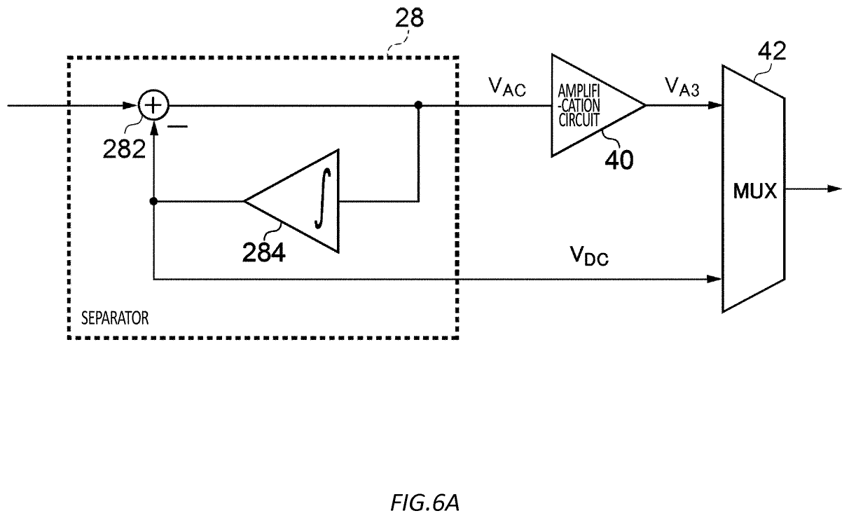

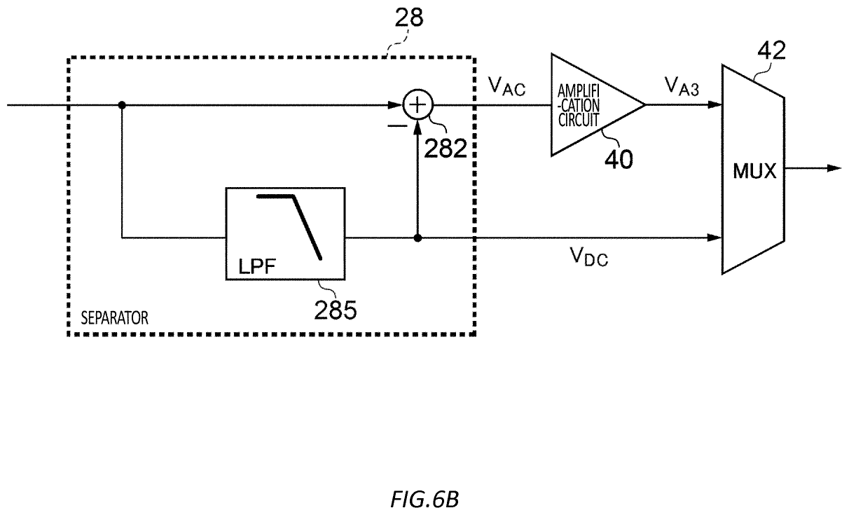

FIGS. 6A and 6B are circuit diagrams illustrating an example of a specific configuration of the separator 28, the amplification circuit 40, and the multiplexer 42. In FIG. 6A, the separator 28 includes an adder 282 and an integrator 284. The separator 28 separates a DC component and other components in the signal VR input to the separator 28 by configuring a so-called DC servo circuit. In FIG. 6B, the separator 28 includes the adder 282 and the low-pass filter 285. The separator 28 separates the DC component and the AC component of VR by extracting only the DC component in the signal VR input to the separator 28 and subtracting the DC component from VR.

Note that, in FIG. 5, a configuration is shown in which signal lines input to the separator 28 are configured in a two-line differential configuration, but in FIGS. 6A and 6B, for convenience of explanation, the separator 28 in a single-ended configuration in which the signal line input to the separator 28 is one line is shown. However, the biological information measurement apparatus 10b including the separator 28 is not limited to the single-ended configuration, and signal lines may be in a two-line fully differential configuration in the entirety of the biological information measurement apparatus 10b.

In FIG. 6A, the integrator 284 integrates the output signal of the adder 282 and applies feedback to a minus input terminal of the adder 282. The input to the separator 28 is a sum of a DC component and an AC component in the vicinity of the DC component, and since an integral value of an AC component having a constant cycle is 0, an integral value of both becomes an addition (integral) value of the DC component, and an output value of the integrator 284 is a value reflecting the DC component. Therefore, by applying feedback of the output of the integrator 284 to the minus input terminal of the adder 282, the separator 28 ultimately outputs the signal VAC from the adder 282 to the amplification circuit 40 as the AC component, and outputs the signal VDC from the integrator 284 to the multiplexer 42 as the DC component.

In FIG. 6B, the low-pass filter 285 blocks only the AC component of the output signal of the adder 282 and applies feedforward to the minus input terminal of the adder 282. Since the input to the separator 28 is a sum of the DC component and the AC component in the vicinity of the DC component, by applying feedforward of the output of the low-pass filter 285 to the minus input terminal of the adder 282, the separator 28 ultimately outputs the signal VAC from the adder 282 to the amplification circuit 40 as the AC component, and outputs the signal VDC from the low-pass filter 285 to the multiplexer 42 as the DC component.

The adder 282 may be configured in a form combining an Op-Amp and the amplification circuit 40 that is an inverting amplifier or a non-inverting amplifier using a resistor or capacitance.

In FIG. 6A, the integrator 284 may have an analog configuration using a resistor, capacitance, and an Op-Amp. Further, the integrator 284 may be configured with an ADC that performs AD (analog-to-digital) conversion of an adder output, a digital arithmetic unit that integrates an AD converter output, and a DAC that performs DA (digital-to-analog) conversion of a digital arithmetic unit output.

In FIG. 6B, the low-pass filter 285 may be an analog filter using a resistor, capacitance, and an Op-Amp. Further, the low-pass filter 285 may be configured with an ADC that performs AD (analog-to-digital) conversion of an adder output, a digital arithmetic unit that blocks an AC component of an AD converter output, and a DAC that performs DA (digital-to-analog) conversion of a digital arithmetic unit output.

Since the amplitude of the signal VAC of the AC component is smaller than the signal VDC of the DC component, the amplification circuit 40 further amplifies the signal VAC that is the AC component and inputs a resultant signal as the signal VA3 to the multiplexer 42. The signal VAC corresponds to the AC component of the signal VR obtained by shifting the bioimpedance component 70 to the vicinity of the zero frequency band, and includes the time-varying component of the bioimpedance component 70.

When the multiplexer 42 is provided as in a configuration of the bioimpedance measurement unit 100b, both the signal VDC of the DC component and the signal VAC of the AC component can be selected and output. The signal VDC of the DC component does not contribute to detection of a component proportional to the motion artifact within the bioimpedance component 70, but by separating and extracting such a DC component VDC, the bioimpedance measurement unit 100b can also measure the average component of the bioimpedance component 70.

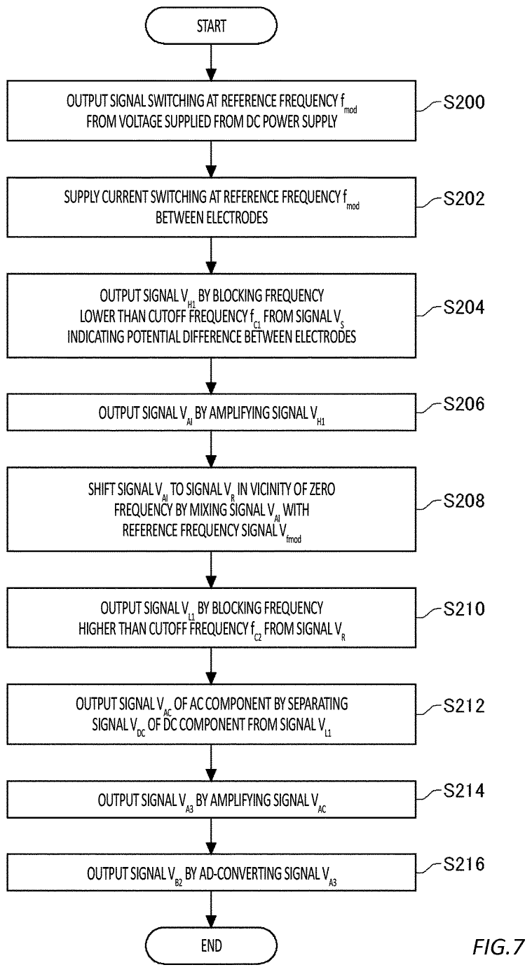

FIG. 7 is an example of a flowchart of a biological information measurement method using the biological information measurement apparatus 10b. The biological information measurement method using the biological information measurement apparatus 10b includes steps S200 to S216.

The chopper 54 outputs, from a DC voltage supplied from the power supply 52 that is a DC power supply, a signal whose voltage level (potential) is switched at the reference frequency fmod (S200). The limiting unit 56 supplies, in accordance with the voltage of the rectangular wave output from the chopper 54, a current between electrodes whose magnitude is switched at the reference frequency fmod (S202).

The bioimpedance measurement unit 100b reads the signal Vs indicating the potential difference generated between the pair of electrodes 150a and 150b that contact the living body 300 when a rectangular-wave current is applied to the pair of electrodes 150a and 150b. The high-pass filter 12 blocks, from the signal VS, a frequency lower than the cutoff frequency fmod and outputs the signal VH1 (S204). The amplification circuit 14 amplifies the signal VH1 and outputs the signal VA1 (S206). The mixer 22 mixes the signal VA1 with the reference frequency signal Vfmod to shift the signal VA1 to the signal VR in the vicinity of the zero frequency (S208).

The low-pass filter 24 blocks, from the signal VR, a frequency higher than the cutoff frequency fC2 and outputs the signal VL1 (S210). The separator 28 separates the DC component VDC and the signal VAC of the AC component of the signal VL1, and outputs the signal VAC of the AC component (S212).

The amplification circuit 40 amplifies the signal VAC and outputs the signal VA3 (S214). The ADC 45 performs AD (analog-to-digital) conversion on the signal VA3 and outputs the signal VB2 (S216).

Even when the configuration of the biological information measurement apparatus 10b is used as the configuration of the biological information measurement apparatus 10, only the time-varying component of the bioimpedance component 70 can be extracted. Further, as described above, according to the biological information measurement apparatus 10b, the component obtained by shifting the average component of the bioimpedance component 70 to the vicinity of the zero frequency can be extracted as the signal VDC from the multiplexer 42. The component is not a portion proportional to a motion artifact that can be noise in biopotential signal measurement in bioimpedance BioZ measurement, but the configuration of the biological information measurement apparatus 10b can be used for applications in measurement using the average value of the bioimpedance BioZ. In the biological information measurement apparatus 10b and the biological information measurement method using the biological information measurement apparatus 10b, the time-varying component of the bioimpedance component 70 with high accuracy can be extracted by an apparatus including an IC with low power consumption and a small area, similarly to the biological information measurement apparatus 10a.

Further, also in the biological information measurement apparatus 10b, in a process of acquiring the signal VAC from the signal VS, a component having a large amplitude other than the time-varying component of the bioimpedance component 70 is excluded from the signal VAC. Therefore, also when the ADC 45 performs AD conversion on the signal VA3, the dynamic range of the ADC 45 is relaxed. Thus, the biological information measurement apparatus 10b can output the signal VB2 obtained by digitally converting a component proportional to the motion artifact while an amplitude of a signal to be subjected to AD conversion relaxes the requirement for the dynamic range of the ADC 45.

Further, by an effect of the mixer 22, the frequency band of the signal VA3 that the ADC 45 performs AD conversion on is shifted to a low-frequency band in the vicinity of the zero frequency. This reduces power consumption for the ADC 45 to perform AD conversion.

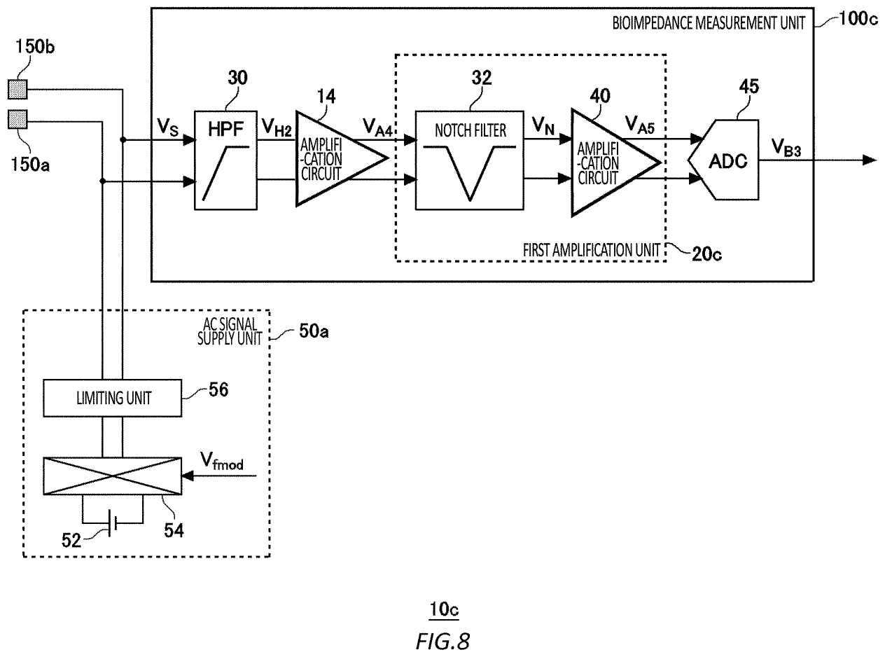

FIG. 8 is an example of a block diagram illustrating a configuration of a biological information measurement apparatus 10c. The biological information measurement apparatus 10c differs from the biological information measurement apparatus 10a and the biological information measurement apparatus 10b in that the biological information measurement apparatus 10c includes a bioimpedance measurement unit 100c.

In the following, description will be given mainly focusing on differences between the bioimpedance measurement unit 100a in FIG. 2 and the bioimpedance measurement unit 100c in FIG. 8. The bioimpedance measurement unit 100c includes a high-pass filter 30, the amplification circuit 14, a first amplification unit 20c, and the ADC 45.

Therefore, the bioimpedance measurement unit 100c differs from the bioimpedance measurement unit 100a in that the bioimpedance measurement unit 100c includes the amplification circuit 14 and the first amplification unit 20c. The high-pass filter 30 may be a filter similar to the high-pass filter 12, but, for convenience of description, the high-pass filter 30 will be described below as a filter having a cutoff frequency fC4 that is higher than a frequency band including the action potential component 72 and lower than the first frequency band. The first amplification unit 20c includes a notch filter 32 and the amplification circuit 40.

In the bioimpedance measurement unit 100c in FIG. 8, similarly to the bioimpedance measurement unit 100a, the signal VS based on the potential difference generated between electrodes 150a and 150b that contact the living body 300 is measured. Next, the high-pass filter 30 having the cutoff frequency fC4 blocks the contribution due to the action potential component 72 from the signal VS.

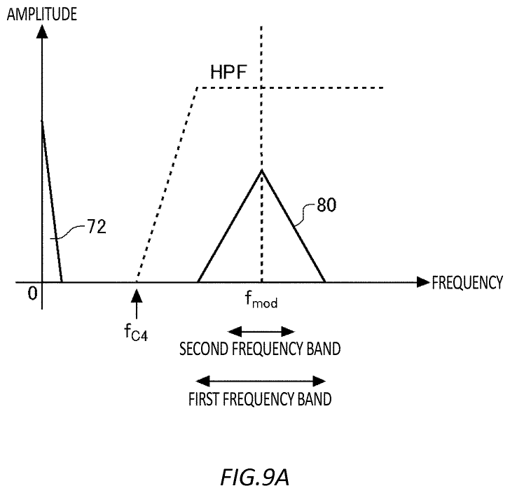

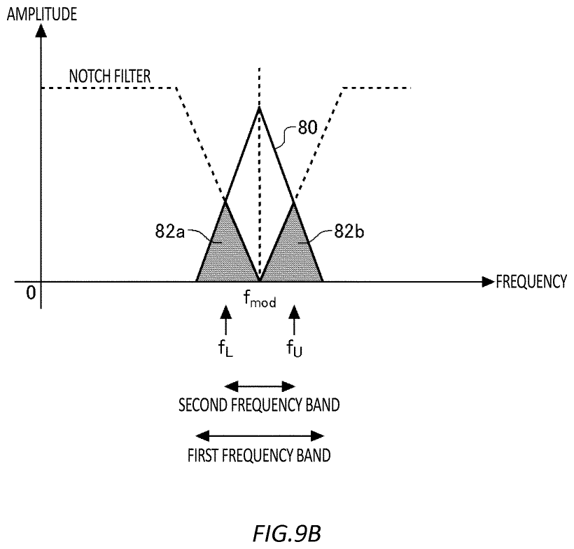

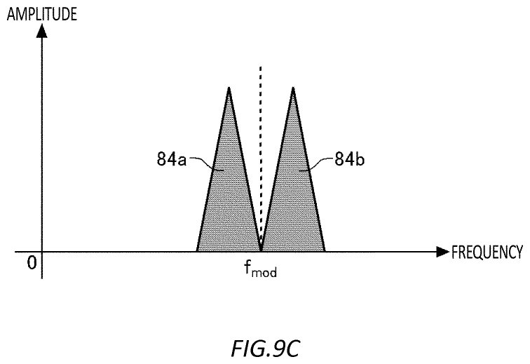

FIG. 9A is an example of a conceptual diagram for explaining an effect of the high-pass filter 30 blocking, from the signal VS, a signal in a band lower than the cutoff frequency fC4. Here, a bioimpedance component 80 has an average component of a high bioimpedance BioZ in the vicinity of the reference frequency fmod, and includes, as a component other than the average value, a time-varying component of the bioimpedance. In the drawing, a frequency band in which the bioimpedance component 80 appears is shown as the first frequency band, and a frequency band in which the average component of the bioimpedance component 80 is included is shown as the second frequency band.

The cutoff frequency fC4 of the high-pass filter 30 is a frequency higher than the contribution from the action potential component 72 and is set to a frequency band lower than the first frequency band. Therefore, after blocking by the high-pass filter 30, the bioimpedance component 80 present in the first frequency band remains. The high-pass filter 30 outputs the signal VH2 corresponding to the bioimpedance component 80.

The amplification circuit 14 in FIG. 8 amplifies the signal VH2 and outputs the signal VA4. In the present embodiment, the signal VH2 is amplified by the amplification circuit 14; however, when the high-pass filter 30 is configured as an active filter, or when a signal in the first frequency band has an amplitude sufficient for processing by subsequent circuits, the amplification circuit 14 may be omitted. The signal VA4 is input to the first amplification unit 20c, and the notch filter 32 blocks, from the signal VA4, a frequency band corresponding to an attenuation band (stop band).

FIG. 9B is an example of a conceptual diagram for explaining an operation in which the notch filter 32 having an attenuation-band lower-limit frequency fL and an attenuation-band upper-limit frequency fU blocks an attenuation band from the signal VA4.

The notch filter 32 blocks the second frequency band from the signal VA4 and outputs a signal VN. Specifically, the notch filter 32 blocks the attenuation band of the attenuation-band lower-limit frequency fL and the attenuation-band upper-limit frequency fU, thereby blocking a high average component appearing in the second frequency band in the vicinity of the reference frequency fmod of the bioimpedance component 80. When the notch filter 32 has a narrow transition width and a steep edge in a frequency band in the vicinity of the lower-limit frequency fL and the upper-limit frequency fU of the attenuation band, the notch filter 32 can block, from the signal VA4, the average component of the bioimpedance component 80 and can extract a time-varying component 82 of the bioimpedance component 80. The notch filter 32 outputs time-varying components 82a and 82b of the bioimpedance component 80 as the signal VN. The signal VN is still another example of the “third signal”.

The amplification circuit 40 in FIG. 8 amplifies the signal VN and outputs a signal VA5. FIG. 9C is an example of a conceptual diagram for explaining the signal VA5 that the amplification circuit 40 outputs by amplifying the signal VN.

The signal VN is the time-varying components 82a and 82b of the bioimpedance component 80. Therefore, the amplification circuit 40 outputs, as the signal VA5, amplified time-varying components 84a and 84b obtained by amplifying the time-varying components 82a and 82b of the bioimpedance component 80.

The ADC 45 in FIG. 8 performs AD (analog-to-digital) conversion on the signal VA5 output from the amplification circuit 40 and outputs a signal VB3. The signal VA5 or the signal VB3 obtained by AD-converting the signal VA5 is still another example of the “biological information signal” indicating the time-varying component of the bioimpedance BioZ of the living body 300.

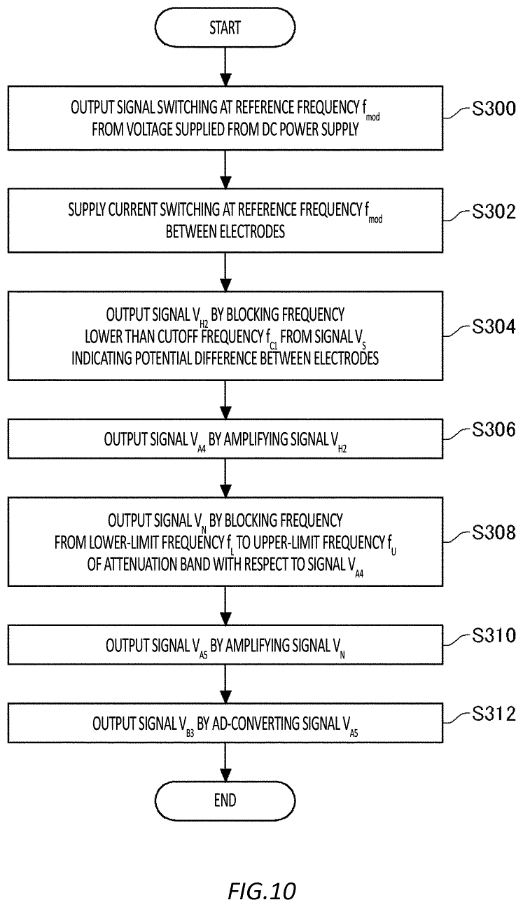

FIG. 10 is a flowchart illustrating an example of a biological information measurement method using the biological information measurement apparatus 10c. The biological information measurement method using the biological information measurement apparatus 10c includes steps S300 to S312. The chopper 54 outputs, from a DC voltage supplied from the power supply 52 that is a DC power supply, a signal whose voltage level (potential) is switched at the reference frequency fmod (S300). The limiting unit 56 supplies, in accordance with the voltage of the rectangular wave output from the chopper 54, a current between electrodes whose magnitude is switched at the reference frequency fmod (S302).

The bioimpedance measurement unit 100c reads the signal Vs indicating the potential difference generated between the pair of electrodes 150a and 150b that contact the living body 300 when a rectangular-wave current is applied to the pair of electrodes 150a and 150b. The high-pass filter 30 blocks, from the signal VS, a frequency lower than the cutoff frequency fmod and outputs the signal VH2 (S304). The amplification circuit 14 amplifies the signal VH2 and outputs the signal VA4 (S306).

The notch filter 32 blocks, from the bioimpedance component 80, a signal in a frequency band higher than the attenuation-band lower-limit frequency fL and lower than the attenuation-band upper-limit frequency fU, and outputs the signal VN (S308). Thus, the bioimpedance measurement unit 100c extracts the time-varying components 82a and 82b of the bioimpedance component 80.

Next, the amplification circuit 40 amplifies the signal VN and outputs the signal VA5 corresponding to the amplified time-varying components 84a and 84b obtained by amplifying the time-varying components 82a and 82b (S310). The ADC 45 performs AD (analog-to-digital) conversion on the signal VA5 and outputs the signal VB3 (S312).

As described above, by configuring the biological information measurement apparatus 10 to have the configuration of the biological information measurement apparatus 10c, or also by the biological information measurement method using the biological information measurement apparatus 10c, only the time-varying component of the bioimpedance component 80 can be extracted. Also in the biological information measurement apparatus 10c, in a process of acquiring the signal VA5 from the signal VS, a large-amplitude signal other than the time-varying component of the bioimpedance component 80 is excluded from the signal VA5. Therefore, even when the ADC 45 AD-converts the signal VA3, it relaxes the requirement for the dynamic range of the ADC 45. Therefore, the biological information measurement apparatus 10c can output the signal VB3 obtained by digitally converting a component proportional to the motion artifact while an amplitude of a signal to be subjected to AD conversion relaxes the requirement for the dynamic range of the ADC 45.

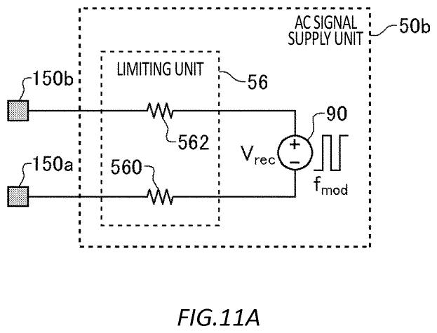

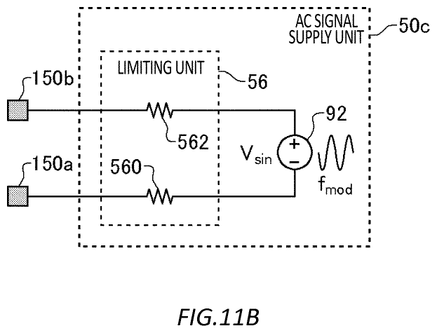

FIG. 11A is a diagram illustrating an example of a configuration of an AC signal supply unit 50b. The AC signal supply unit 50b includes a power supply 90 and the limiting unit 56.

The power supply 90 is a power supply that outputs a rectangular-wave voltage Vsin having the reference frequency fmod. As in the present embodiment, in an AC signal supply unit 50a, the power supply 52 that outputs a DC voltage and the chopper 54 that modulates a signal of the DC voltage output by the power supply 52 into a rectangular-wave signal having the reference frequency fmod may be replaced with the power supply 90. In this manner, the AC signal supply unit 50b, unlike the AC signal supply unit 50a, functions as a source of an AC signal without modulation by the chopper 54.

The limiting unit 56 limits the current value applied to the electrodes 150a and 150b. The limiting unit 56 includes resistors 560 and 562. Note that a resistance value of the resistor 560 may be a same value as that of the resistor 562, and may be set to a different value as desired.

FIG. 11B is a diagram illustrating an example of a configuration of an AC signal supply unit 50c. The AC signal supply unit 50c includes a power supply 92 and the limiting unit 56.

The power supply 92 is a power supply that outputs a sinusoidal voltage Vsin. As in the present embodiment, in the AC signal supply unit 50c, the power supply 52 and the chopper 54 in the AC signal supply unit 50a may be replaced with the power supply 92. In this manner, the AC signal supply unit 50c, unlike the AC signal supply unit 50a, functions as a source of an AC signal without modulation by the chopper 54, and supplies a sinusoidal AC signal. As in the present embodiment, each biological information measurement apparatus 10 may read the bioimpedance BioZ from a current flowing through the living body 300 via the electrodes 150a and 150b when the sinusoidal AC signal is supplied.

The limiting unit 56 limits the current value applied to the electrodes 150a and 150b. A configuration of the limiting unit 56 may be similar to that of the AC signal supply unit 50b.

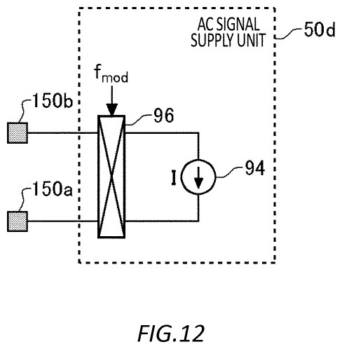

FIG. 12 is a diagram illustrating an example of a configuration of an AC signal supply unit 50d. The AC signal supply unit 50d includes a current source 94 and a mixer 96.

The AC signal supply unit 50d corresponds to a configuration in which the power supply 52 that is a voltage source in the AC signal supply unit 50a is replaced with the current source 94 and the limiting unit 56 is omitted. Thus, each of the power supplies 52, 90, and 92 that are voltage sources including the AC signal supply units 50a to 50c may be replaced with the current source 94.

In any of the methods of the AC signal supply units 50a to 50d described above, in order to provide insulation for the DC component between the AC signal supply units 50a to 50d and the living body 300, the AC signal supply units 50a to 50d may be connected to the electrodes 150a and 150b via capacitance such as a capacitor.

Here, in the AC signal supply units 50a to 50c, any of the power supplies 52, 90, and 92 that are voltage sources is used. In this case, the bioimpedance measurement unit 100 measures the variation of the bioimpedance BioZ by measuring the signal Vs indicating a potential difference between the electrodes 150a and 150b. Furthermore, as will be described later with reference to FIG. 14, by providing a biopotential signal output unit 200 connected to the electrodes 150a and 150b, a biopotential signal with reduced influence of MA can be output.

In the AC signal supply unit 50 that applies an AC signal to a living body, when any of the power supplies 52, 90, and 92 that are voltage sources is used and both the biopotential signal and the time-varying component of the bioimpedance are measured, the biopotential signal can appear as a relatively low-frequency component regardless of the frequency of the AC signal. Therefore, the low-frequency component of noise included in the AC signal may become measurement noise of the biopotential signal. The capacitance that reduces the DC component of the AC signal can reduce such noise. Furthermore, by voltage division between the bioimpedance and the resistors 560 and 562 and the capacitance for limiting current in the limiting unit 56, it is possible to suppress noise originating from the AC signal supply unit 50 from being superimposed on a band of the biopotential signal (for example, a band of 20 Hz or higher and lower than 4 kHz when the biopotential signal is a myoelectric signal).

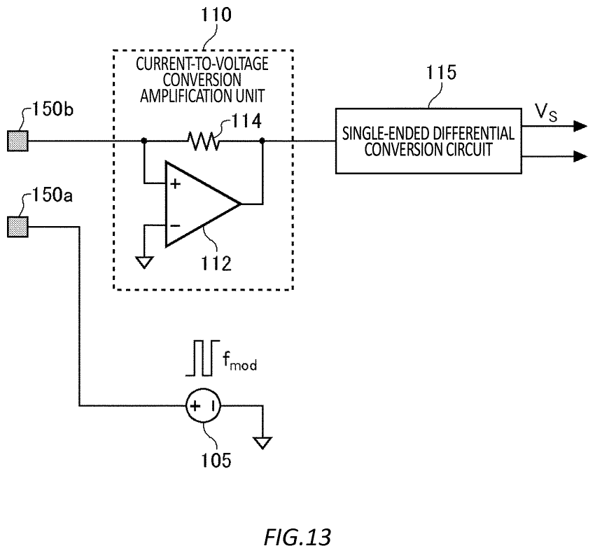

FIG. 13 is a diagram illustrating an example of a modification in which components other than the bioimpedance measurement unit 100a in the embodiment of FIG. 2 are replaced. The biological information measurement apparatus 10 includes a power supply 105, a current-to-voltage conversion amplification unit 110, and a single-ended differential conversion circuit 115.

The power supply 105 supplies a single-ended rectangular-wave voltage signal with reference to a voltage at the electrode 150a. Therefore, the circuit connected to the electrode 150a corresponds to an “AC signal output circuit” including the power supply 105 corresponding to the “voltage output circuit”.

The current-to-voltage conversion amplification unit 110 converts a current flowing through the living body 300 via the pair of electrodes 150a and 150b into a voltage signal, and amplifies the converted voltage signal. The current-to-voltage conversion amplification unit is connected to the electrode 150b. The current-to-voltage conversion amplification unit 110 includes an amplification circuit 112 and a resistor 114.

The amplification circuit 112 is, for example, an Op-Amp. The resistor 114 converts the current signal supplied from the electrode 150b into a voltage signal. The amplification circuit 112 and the resistor 114 constitute a non-inverting amplifier circuit, and convert the output voltage signal into an amplified signal. Here, the circuit including the amplification circuit 112 and the resistor 114 is not limited to the non-inverting amplifier circuit, and an inverting amplifier circuit may be used.

The single-ended differential conversion circuit 115 converts the single-ended voltage signal output by the current-to-voltage conversion amplification unit 110 into the signal VS that is a differential signal. The single-ended differential conversion circuit 115 is a circuit that includes, for example, a differential amplifier, level-shifts a single-ended signal, and outputs a differential signal. The signal VS may be supplied to any of the bioimpedance measurement units 100a to 100c. By using the single-ended differential conversion circuit 115, even when a current is caused to flow from the single-ended power supply 105 to the electrode 150a and a single-ended current signal is extracted from the electrode 150b, the signal VS that is a differential voltage signal can be obtained.

FIG. 14 is the block diagram illustrating an example of a configuration of a biological information measurement apparatus 10d. The biological information measurement apparatus 10d includes, in addition to the AC signal supply unit 50, the bioimpedance measurement unit 100, and the electrode 150, a generation unit 60 and the biopotential signal output unit 200. The bioimpedance measurement unit 100 may be any of the bioimpedance measurement units 100a, 100b, and 100c that have already been described.

The bioimpedance measurement unit 100 outputs the signal VB1 proportional to the time-varying portion of the bioimpedance BioZ from a signal based on a potential generated between the electrodes 150a and 150b.

The generation unit 60 outputs a signal VMA indicating the motion artifact in accordance with the signal VB1. The generation unit 60 may generate the signal VMA and output the signal VMA based on relationship information indicating a relationship between the signal VB1 and the signal VMA and the signal VB1 input from the bioimpedance measurement unit 100. The generation unit 60 may generate the signal VMA from the signal VB1 input from the bioimpedance measurement unit 100 and output the signal VMA in accordance with a predetermined function that receives the signal VB1 as an input and outputs the signal VMA. The generation unit 60 may generate the signal VMA by multiplying the signal VB1 input from the bioimpedance measurement unit 100 by a predetermined coefficient, and output the signal VMA. In the measurement of electromyography, the motion artifact corresponds to a noise component included in a component in which action potentials from each of a plurality of muscle fibers constituting the living body 300 are combined. Therefore, the generation unit 60 generates the noise signal VMA indicating such a noise component based on the signal VB1.

The biopotential signal output unit 200 reads a biopotential from the potential generated between the electrodes 150a and 150b, and outputs the signal VEMG corresponding to the biopotential. The biopotential signal output unit 200 removes the noise signal VMA corresponding to the motion artifact from a signal VEMG+MA corresponding to the measured biopotential. Thus, for example, when the biopotential signal to be measured is a myoelectric signal, the biopotential signal output unit 200 outputs the biopotential signal VEMG as a myoelectric signal indicating a component in which action potentials from each of a plurality of muscle fibers 308 constituting the living body 300 are combined. The biopotential signal output unit 200 includes a second amplification unit 210, an ADC 220, and a subtractor 222.

The second amplification unit 210 blocks, from the signal Vs, a frequency band lower than the fifth frequency band including a component in which action potentials from each of a plurality of biological tissues constituting the living body 300 are combined, and a frequency band higher than the fifth frequency band and including the first frequency band, and amplifies a signal in the fifth frequency band. The second amplification unit 210 includes the high-pass filter 212, an amplification circuit 214, and a low-pass filter 216.

The biopotential signal output unit 200 measures the signal VS indicating the potential difference generated between the electrodes 150a and 150b that contact the living body 300. The high-pass filter 212 blocks a frequency band lower than the fifth frequency band from the signal VS, and outputs a signal VH3. A cutoff frequency of the high-pass filter 212 is, for example, 20 Hz similarly to the high-pass filter 26. Thus, the high-pass filter 212 blocks, from the signal VS, a contribution due to the half-cell potential component 73, and outputs the signal VH3 including the residual component 74 including a component in which action potentials from each of a plurality of biological tissues (for example, the muscle fibers 308) constituting the living body 300 are combined, and the bioimpedance component 70.

The amplification circuit 214 amplifies the signal VH3 and outputs the signal VA5. The high-pass filter 212 may be an active filter that blocks, from the signal VS, the contribution due to the half-cell potential component 73 and also amplifies the signal VS, and in this case, the amplification circuit 214 may be omitted.

The low-pass filter 216 blocks the bioimpedance component 70 from the signal VA5, and functions as an anti-aliasing filter for preventing generation of a high-frequency aliasing signal in AD conversion of an analog signal. The low-pass filter 216 blocks the high-frequency band of the signal VA5 and outputs a signal VL2. The low-pass filter 216 blocks a frequency band that is higher than the fifth frequency band and includes the first frequency band. The low-pass filter 216 may be an active filter that blocks and amplifies a high-frequency band of the signal VA5.

Note that in this case, the low-pass filter 216 only needs to be capable of blocking the first frequency band and to have a cutoff frequency at a frequency higher than an upper limit of the action potential component 72. In other words, the upper limit of the fifth frequency band only needs to be lower than a lower limit of the first frequency band and to be a frequency that is equal to or higher than the upper limit of the action potential component 72 of the biopotential signal to be measured, and only needs to be a value that is equal to or higher than an upper limit of a desired biopotential signal. Therefore, the upper limit of the fifth frequency band may be set to, for example, 4 kHz or higher when a myoelectric signal is measured as the biopotential signal, may be set to 5 kHz or higher, or may be set to 10 kHz or higher. As another example, the upper limit of the fifth frequency band may be set to, for example, 100 Hz or higher when an electrocardiographic signal is to be measured, or may be set to 200 Hz or higher. In this case, the cutoff frequency of the low-pass filter 216 may be set to a frequency that is 100 Hz or higher or higher than 200 Hz and lower than the lower limit of the first frequency band.

The ADC 220 AD-converts the signal VL2 and outputs the signal VEMG+MA. Therefore, the ADC 220 AD-converts the signal VL2 corresponding to the residual component 74 including a component in which action potentials from each of the muscle fibers 308 are combined, and outputs the signal VEMG+MA.

The subtractor 222 outputs the signal VEMG, which is a biopotential signal with less influence of motion artifacts, by subtracting, from the signal VEMG+MA, the component of the noise signal VMA indicating the motion artifact. For example, when the biopotential signal to be measured is a myoelectric signal, the biopotential signal output unit 200 of the biological information measurement apparatus 10d can output the biopotential signal VEMG that is a myoelectric signal from which an influence of motion artifacts has been removed.

According to the configuration of the biological information measurement apparatus 10d of the present embodiment, for example, when the biopotential signal to be measured is a myoelectric signal, the biological information measurement apparatus 10d can acquire, with high accuracy, the biopotential signal VEMG for generating a surface electromyogram (Electro Myo Graphy) in which action potentials from each of a plurality of biological tissues (for example, the muscle fibers 308) constituting the living body 300 are combined. In addition, the biological information measurement apparatus 10d can realize configurations of a low-power-consumption analog front end and the ADC 45 with a low-area IC, and can exclude noise caused by motion artifacts from the biopotential signal VEMG such as a myoelectric signal.

The high-pass filter 212 is an example of the “third high-pass filter”. The amplification circuit 214 is an example of the “third amplification circuit”. The low-pass filter 216 is an example of the “second low-pass filter”. The signal VE is an example of the “biopotential signal”.

While the present invention has been described by way of the embodiments, the technical scope of the present invention is not limited to the above-described embodiments. It is apparent to persons skilled in the art that various alterations or improvements can be added to the above-described embodiments. It is also apparent from the described scope of the claims that the embodiments added with such alterations or improvements can be included the technical scope of the present invention.

Note that the operations, procedures, steps, stages, or the like of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

### OTHER POSSIBLE ITEMS

### Item 1

A biological information measurement apparatus including:
an AC signal supply unit that supplies an AC signal indicating a reference frequency between a pair of electrodes that contact a living body;
a first high-pass filter that blocks, from a first signal corresponding to the AC signal, a frequency band lower than a first frequency band including the reference frequency; and
a first amplification unit that amplifies a third signal, which is obtained by blocking, from a second signal output from the first high-pass filter, a second frequency band narrower than the first frequency band and including the reference frequency, and outputs a resultant signal as a biological information signal indicating a time-varying component of impedance of the living body.

### Item 2

The biological information measurement apparatus according to item 1, wherein
the first amplification unit includes an amplification circuit that amplifies the third signal and outputs a resultant signal as the biological information signal, and
the third signal is a signal corresponding to a component of the second signal that is within the first frequency band and outside the second frequency band.

### Item 3

The biological information measurement apparatus according to item 2, wherein
the first amplification unit includes
a first mixer that converts, by mixing an AC signal indicating the reference frequency with the second signal, the second signal into a signal in a third frequency band lower than the first frequency band,
a first low-pass filter that blocks, from the signal output from the first mixer, a fourth frequency band higher than the third frequency band and including the reference frequency, and
a second high-pass filter that blocks, from a signal output from the first low-pass filter, a frequency band lower than a first frequency in the third frequency band and outputs a resultant signal as the third signal.

### Item 4

The biological information measurement apparatus according to item 2, wherein
the first amplification unit includes
a first mixer that converts, by mixing an AC signal indicating the reference frequency with the second signal, the second signal into a signal in a third frequency band lower than the first frequency band,
a first low-pass filter that blocks, from the signal output from the first mixer, a fourth frequency band higher than the third frequency band and including the reference frequency, and
a separator that separates the signal output from the first low-pass filter into a signal of a component in a frequency band lower than a first frequency in the third frequency band and a signal of a component in a frequency band higher than the first frequency, and outputs, as the third signal, the signal of the component in the frequency band higher than the first frequency.

### Item 5

The biological information measurement apparatus according to item 2, wherein
the first amplification unit includes
a notch filter that blocks the second frequency band from the second signal and outputs a resultant signal as the third signal.

### Item 6

The biological information measurement apparatus according to item 1, wherein
the first signal includes a biopotential component in which action potentials from each of a plurality of biological tissues constituting the living body are combined, a time-varying component of impedance of the living body, and an average component of impedance of the living body.

### Item 7

The biological information measurement apparatus according to item 6, wherein
the first amplification unit removes the average component of impedance of the living body by blocking the second frequency band from the second signal.

### Item 8

The biological information measurement apparatus according to item 1, wherein
the AC signal supply unit includes
a voltage output circuit that outputs an AC voltage signal indicating the reference frequency, and
a limiting unit that limits an amount of current flowing from the voltage output circuit through the living body via the pair of electrodes, and
the first signal indicates a potential difference between the pair of electrodes generated by the current flowing through the living body.

### Item 9

The biological information measurement apparatus according to item 1, wherein
the AC signal supply unit includes
a current output circuit that outputs an AC current signal indicating the reference frequency, and
the first signal indicates a potential difference between the pair of electrodes generated by the current flowing through the living body.

### Item 10

The biological information measurement apparatus according to item 1, wherein
the AC signal supply unit includes a voltage output circuit that outputs an AC voltage signal indicating the reference frequency, and
the biological information measurement apparatus includes: a current-to-voltage conversion amplification unit that converts current flowing through the living body via the pair of electrodes into a voltage signal; and
a single-ended differential conversion circuit that outputs, as the first signal, a signal obtained by single-ended differential conversion of the voltage signal.

### Item 11

The biological information measurement apparatus according to any one of items 1 to 10,further including:
a generation unit that generates, based on the biological information signal, a noise signal indicating a noise component included in a component in which action potentials from each of a plurality of biological tissues constituting the living body are combined; and
a biopotential signal output unit that removes a component of the noise signal from the first signal and outputs a resultant signal as a biopotential signal indicating a component in which action potentials from each of a plurality of muscle fiber biological tissues constituting the living body are combined.

### Item 12

The biological information measurement apparatus according to item 11, wherein
the biopotential signal output unit includes
a second amplification unit that blocks, from the first signal, a frequency band lower than a fifth frequency band including a component in which action potentials from each of a plurality of biological tissues constituting the living body are combined and a frequency band higher than the fifth frequency band and including the first frequency band, and amplifies a signal in the fifth frequency band, and
the biopotential signal output unit outputs the biopotential signal by removing the component of the noise signal from the signal output from the second amplification unit.

### Item 13

A biological information measurement method including:
supplying an AC signal indicating a reference frequency between a pair of electrodes that contact a living body;
blocking, from a first signal corresponding to the AC signal, a frequency band lower than a first frequency band including the reference frequency and outputting a second signal;
blocking, from the second signal, a second frequency band narrower than the first frequency band and including the reference frequency and outputting a third signal; and
amplifying the third signal and outputting a resultant signal as a biological information signal indicating a time-varying component of impedance of the living body.

### EXPLANATION OF REFERENCES

10: biological information measurement apparatus;
12, 26, 30, 212: high-pass filter;
14, 40, 112, 214: amplification circuit;
20: first amplification unit;
22, 96: mixer;
24, 216: low-pass filter;
28: separator;
32: notch filter;
42: multiplexer;
45, 220: ADC;
50: AC signal supply unit;
52, 90, 92, 105: power supply;
54: chopper;
56: limiting unit;
60: generation unit;
70, 80: impedance component;
72: action potential component;
73: half-cell potential component;
74: residual component;
75: shift residual component;
77: high-frequency shift component;
78: frequency band;
79, 82: time-varying component;
84: amplified time-varying component;
94: current source;
100: bioimpedance measurement unit;
110: current-to-voltage conversion amplification unit;
114, 560, 562: resistor;
115: single-ended differential conversion circuit;
150: electrode;
200: biopotential signal output unit;
222: subtractor;
282: adder;
284: integrator;
285: low-pass filter;
300: living body;
302: epidermal layer;
304: dermis and subcutaneous layer;
306: muscle layer;
308: muscle fiber; and
310: nerve.

What is claimed is:

1. A biological information measurement apparatus comprising:
an AC signal supply unit that supplies an AC signal indicating a reference frequency between a pair of electrodes that contact a living body;
a first high-pass filter that blocks, from a first signal corresponding to the AC signal, a frequency band lower than a first frequency band including the reference frequency; and
a first amplification unit that amplifies a third signal, which is obtained by blocking, from a second signal output from the first high-pass filter, a second frequency band narrower than the first frequency band and including the reference frequency, and outputs a resultant signal as a biological information signal indicating a time-varying component of impedance of the living body.

2. The biological information measurement apparatus according to claim 1, wherein
the first amplification unit includes an amplification circuit that amplifies the third signal and outputs a resultant signal as the biological information signal, and
the third signal is a signal corresponding to a component of the second signal that is within the first frequency band and outside the second frequency band.

3. The biological information measurement apparatus according to claim 2, wherein
the first amplification unit includes
a first mixer that converts, by mixing an AC signal indicating the reference frequency with the second signal, the second signal into a signal in a third frequency band lower than the first frequency band,
a first low-pass filter that blocks, from the signal output from the first mixer, a fourth frequency band higher than the third frequency band and including the reference frequency, and
a second high-pass filter that blocks, from a signal output from the first low-pass filter, a frequency band lower than a first frequency in the third frequency band and outputs a resultant signal as the third signal.

4. The biological information measurement apparatus according to claim 2, wherein
the first amplification unit includes
a first mixer that converts, by mixing an AC signal indicating the reference frequency with the second signal, the second signal into a signal in a third frequency band lower than the first frequency band,
a first low-pass filter that blocks, from the signal output from the first mixer, a fourth frequency band higher than the third frequency band and including the reference frequency, and
a separator that separates the signal output from the first low-pass filter into a signal of a component in a frequency band lower than a first frequency in the third frequency band and a signal of a component in a frequency band higher than the first frequency, and outputs, as the third signal, the signal of the component in the frequency band higher than the first frequency.

5. The biological information measurement apparatus according to claim 2, wherein
the first amplification unit includes
a notch filter that blocks the second frequency band from the second signal and outputs a resultant signal as the third signal.

6. The biological information measurement apparatus according to claim 1, wherein
the first signal includes a biopotential component in which action potentials from each of a plurality of biological tissues constituting the living body are combined, a time-varying component of impedance of the living body, and an average component of impedance of the living body.

7. The biological information measurement apparatus according to claim 3, wherein
the first signal includes a biopotential component in which action potentials from each of a plurality of biological tissues constituting the living body are combined, a time-varying component of impedance of the living body, and an average component of impedance of the living body.

8. The biological information measurement apparatus according to claim 4, wherein
the first signal includes a biopotential component in which action potentials from each of a plurality of biological tissues constituting the living body are combined, a time-varying component of impedance of the living body, and an average component of impedance of the living body.

9. The biological information measurement apparatus according to claim 5, wherein
the first signal includes a biopotential component in which action potentials from each of a plurality of biological tissues constituting the living body are combined, a time-varying component of impedance of the living body, and an average component of impedance of the living body.

10. The biological information measurement apparatus according to claim 6, wherein
the first amplification unit removes the average component of impedance of the living body by blocking the second frequency band from the second signal.

11. The biological information measurement apparatus according to claim 7, wherein
the first amplification unit removes the average component of impedance of the living body by blocking the second frequency band from the second signal.

12. The biological information measurement apparatus according to claim 8, wherein
the first amplification unit removes the average component of impedance of the living body by blocking the second frequency band from the second signal.

13. The biological information measurement apparatus according to claim 9, wherein
the first amplification unit removes the average component of impedance of the living body by blocking the second frequency band from the second signal.

14. The biological information measurement apparatus according to claim 1, wherein
the AC signal supply unit includes
a voltage output circuit that outputs an AC voltage signal indicating the reference frequency, and
a limiting unit that limits an amount of current flowing from the voltage output circuit through the living body via the pair of electrodes, and
the first signal indicates a potential difference between the pair of electrodes generated by the current flowing through the living body.

15. The biological information measurement apparatus according to claim 1, wherein
the AC signal supply unit includes
a current output circuit that outputs an AC current signal indicating the reference frequency, and
the first signal indicates a potential difference between the pair of electrodes generated by the current flowing through the living body.

16. The biological information measurement apparatus according to claim 1, wherein
the AC signal supply unit includes a voltage output circuit that outputs an AC voltage signal indicating the reference frequency, and
the biological information measurement apparatus comprises: a current-to-voltage conversion amplification unit that converts current flowing through the living body via the pair of electrodes into a voltage signal; and
a single-ended differential conversion circuit that outputs, as the first signal, a signal obtained by single-ended differential conversion of the voltage signal.

17. The biological information measurement apparatus according to claim 1, further comprising:
a generation unit that generates, based on the biological information signal, a noise signal indicating a noise component included in a component in which action potentials from each of a plurality of biological tissues constituting the living body are combined; and
a biopotential signal output unit that removes a component of the noise signal from the first signal and outputs a resultant signal as a biopotential signal indicating a component in which action potentials from each of a plurality of muscle fiber biological tissues constituting the living body are combined.

18. The biological information measurement apparatus according to claim 6, further comprising:
a generation unit that generates, based on the biological information signal, a noise signal indicating a noise component included in a component in which action potentials from each of a plurality of biological tissues constituting the living body are combined; and
a biopotential signal output unit that removes a component of the noise signal from the first signal and outputs a resultant signal as a biopotential signal indicating a component in which action potentials from each of a plurality of muscle fiber biological tissues constituting the living body are combined.

19. The biological information measurement apparatus according to claim 17, wherein
the biopotential signal output unit includes
a second amplification unit that blocks, from the first signal, a frequency band lower than a fifth frequency band including a component in which action potentials from each of a plurality of biological tissues constituting the living body are combined and a frequency band higher than the fifth frequency band and including the first frequency band, and amplifies a signal in the fifth frequency band, and
the biopotential signal output unit outputs the biopotential signal by removing the component of the noise signal from the signal output from the second amplification unit.

20. A biological information measurement method comprising:
supplying an AC signal indicating a reference frequency between a pair of electrodes that contact a living body;
blocking, from a first signal corresponding to the AC signal, a frequency band lower than a first frequency band including the reference frequency and outputting a second signal;
blocking, from the second signal, a second frequency band narrower than the first frequency band and including the reference frequency and outputting a third signal; and
amplifying the third signal and outputting a resultant signal as a biological information signal indicating a time-varying component of impedance of the living body.
